(19) United States
(12) Patent Application Publication
(10) Pub. No.: US 2013/0267558 A1
(43) Pub. Date: Oct. 10, 2013

(54) PHARMACEUTICAL COMPOSITION FOR TREATING OR PREVENTING ALCOHOLIC LIVER DISEASES, CONTAINING CILOSTAZOL AS ACTIVE INGREDIENT

(71) Applicants: Jong-Yeon Kim, Daegu (KR); Jong-Ryul Eun, Daegu (KR); Youn-Ju Lee, Daegu (KR)

(72) Inventors: Jong-Yeon Kim, Daegu (KR); Jong-Ryul Eun, Daegu (KR); Youn-Ju Lee, Daegu (KR)

(73) Assignee: INDUSTRY-ACADEMIC COOPERATION FOUNDATION YEUNGNAM UNIVERSITY, GYEONGSANGBUK-DO (KR)

(21) Appl. No.: 13/880,792

(22) PCT Filed: Oct. 7, 2011
(86) PCT No.: PCT/KR11/07441

§ 371 Date: Jun. 26, 2013

(30) Foreign Application Priority Data
Oct. 22, 2010 (KR) 10-2010-0103656

(51) Int. Cl. A61K 31/4709

(52) U.S. Cl. CPC A61K 31/4709
USPC 514/312; 546/158

(57) ABSTRACT

Provided is a pharmaceutical composition for the treatment and prevention of alcoholic liver diseases, including cilostazol as an active ingredient. Cilostazol inhibits expression levels of TNF-α and FAS (fatty acid synthase) gene in a concentration-dependent manner, and also significantly inhibits the activity of caspase-3. Accordingly, cilostazol shows superior effects for the treatment or prevention of alcoholic liver diseases, in particular, alcoholic hepatitis compared to pentoxifylline which is conventionally used as a therapeutic agent for the treatment for alcoholic hepatitis. Thus, cilostazol is suitable for use as a drug for the treatment or prevention of alcoholic hepatitis.

### TECHNICAL FIELD

The present invention relates to a pharmaceutical composition including cilostazol as an active ingredient for the treatment and prevention of alcoholic liver diseases, in particular, alcoholic hepatitis.

### BACKGROUND ART

Alcoholic liver disease is prevalent throughout the world, and even in South Korea, heavy alcohol drinking is an issue. Alcoholic liver disease can be classified as alcoholic fatty liver (steatosis), alcoholic hepatitis and alcoholic liver cirrhosis. Alcohol abuse increases the synthesis of triglyceride leading to fat accumulation in the liver cells in which alcohol metabolites, acetaldehyde and acetate play roles. Fatty liver is reversible within a few weeks after alcohol intake has completely stopped. Continuous alcohol drinking, however, can cause inflammation together with steatosis to develop fatty hepatitis which can further progress to fibrosis. Mechanisms proposed to be important for the pathogenesis of alcoholic liver damage include oxidative stress, immune reaction, and secretion of various cytokines.

Alcoholic hepatitis occurs via mechanisms different from that for alcoholic fatty liver. Alcoholic hepatitis is a type of an acute hepatitis that occurs mostly due to binge drinking, i.e., drinking too much alcohol in a short period of time. Heavy drinking increases the permeability of intestinal mucosa resulting in the increased passage of endotoxin, gram-negative bacterial cell wall products (that is, lipopolysaccharide, LPS), through the intestinal wall into the bloodstream of portal vein. Upon reaching the liver, endotoxin binds to a particular receptor (Toll-like receptor 4) located on the surface of Kupffer cells, activating intracellular signaling pathways. Kupffer cells, in turn, release cytokines including TNF-α that regulates apoptosis, necrosis, and inflammation of liver cells.

In the condition of alcoholic fatty liver, oxidative stress contributes to the liver cell damage, which can be completely recovered by just stopping alcohol drinking, therefore anti-oxidants may be used as auxiliary therapeutic agents. Although total alcohol abstinence may be enough for treatment of mild alcoholic hepatitis, severe alcoholic hepatitis can be life-threatening and about 40 to 50% of patients may die without therapeutic intervention. Treatment of severe alcoholic hepatitis requires suppression of inflammatory response. For this purpose, steroids and pentoxifylline, inhibitors of systemic inflammation and TNFalpha, respectively, are currently approved in clinical trials. Since steroids have many adverse effects, penotixifylline is now preferred for the treatment of severe alcoholic hepatitis. However, therapeutic effects of pentoxifylline are not satisfactory.

Cilostazol approved by US FDA in 1999 is widely used for various kinds of vascular diseases including atherosclerosis. Cilostazol increases the intracellular level of 3′,5′-cyclic adenosine monophosphate (cAMP) by inhibiting phosphodiesterase-3 (PDE-3). However, effects of cilostazol on alcoholic liver diseases, in particular, alcoholic hepatitis have not been reported.

### DETAILED DESCRIPTION OF THE INVENTION

### Technical Problem

To resolve such problems, the inventors of the present application evaluated therapeutic and preventive effects of cilostazol on alcoholic liver diseases, in particular, alcoholic hepatitis, and found that cilostazol inhibited TNF-α production more potently than pentoxifylline which is widely used for severe alcoholic hepatitis. In addition, cilostazol prevented the liver cell damage caused by alcohol consumption in in vivo model, thereby completing the present invention.

Accordingly, the present invention provides a pharmaceutical composition including cilostazol or a pharmaceutically acceptable salt thereof as an active ingredient for the treatment and prevention of alcoholic liver diseases.

### Technical Solution

To achieve this goal, the present invention provides a pharmaceutical composition including cilostazol or a pharmaceutically acceptable salt thereof for the treatment and prevention of alcoholic liver diseases. The structure of cilostazol is represented in Formula 1:

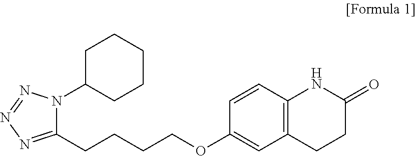 [SMILES: O=C1CCc2cc(OCCCCc3nnnn3C3CCCCC3)ccc2N1]

The cilostazol or pharmaceutically acceptable salt thereof is a TNF-α inhibitor which inhibits production of TNF-α induced by LPS, expression of fatty acid synthase (FAS) gene, and caspase-3 activity in vitro and in vivo.

The alcoholic liver diseases may be alcoholic hepatitis.

The pharmaceutically acceptable salt of cilostazol may be in the form of an acid addition salt. For example, the pharmaceutically acceptable salt of cilostazol may be easily prepared by reacting cilostazol with a pharmaceutically acceptable acid. The pharmaceutically acceptable acid may be, for example, an organic acid, such as an oxalic acid, a maleic acid, a fumaric acid, a malic acid, a tartaric acid, a citric acid, or a benzoic acid, or an inorganic acid, such as a hydrochloric acid, a sulfuric acid, a phosphoric acid, or a hydrobromic acid.

According to an embodiment of the present invention, cilostazol or pharmaceutically acceptable salt thereof may be prepared in one formulation selected from a powder formulation, a tablet formulation, a capsule formulation, an injection formulation, or an aerosol.

According to the present invention, cilostazol or pharmaceutically acceptable salt thereof may be included in the pharmaceutical composition in an amount of 0.1 to 50 parts by weight based on 100 parts by weight of the pharmaceutical composition. When cilostazol or pharmaceutically acceptable salt thereof is included in an amount of less than 0.1 parts by weight, pharmaceutical effects thereof are negligible. When cilostazol or pharmaceutically acceptable salt thereof is included in an amount of greater than 50 parts by weight, pharmaceutical effects may be saturated, which is not economical, and also, adverse effects may occur.

Also, the pharmaceutical composition according to the present invention may further include appropriate carriers, excipient, or diluents which are conventionally used in preparing pharmaceutical compositions.

Examples of carriers, excipient, or diluents that are available for use in the pharmaceutical composition in the present invention are lactose, dextrose, sucrose, sorbitol, mannitol, xylitol, erythritol, maltitol, starch, Acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils.

The pharmaceutical composition according to the present invention may be prepared into an oral formulation such as a powder formulation, a granule formulation, a tablet formulation, a capsule formulation, a suspension formulation, an emulsion formulation, a syrup formulation, or an aerosol formulation, an external formulation, a suppository formulation, or a sterilized injection solution formation, according to conventional methods.

When prepared into various formulations, a conventional diluent or excipient, such as a filler, a bulking agent, a binding agent, a wetting agent, an disintegrating agent or a surfactant, may be used. A solid formulation for oral administration may be a tablet formulation, a pill formulation, a powder formulation, a granule formulation, or a capsule formulation, and such solid formulations are prepared by mixing one or more excipients, for example, starch, calcium carbonate, sucrose, lactose, or gelatin.

Also, in addition to such excipients, a lubricating agent such as magnesium stearate or talc may be used. A liquid formulation for oral administration may be a suspension formulation, an internal solution formulation, an oil formulation, or a syrup formulation. The liquid formulation may include various excipients, for example, a wetting agent, a sweetening agent, a perfuming agent, or a preservative, in addition to a conventional diluent such as water or liquid paraffin.

A formulation for non-oral administration may be a sterilized aqueous solution formulation, a non-aqueous solution formulation, a suspension formulation, an oil formulation, a lyophilized formulation, or a suppository formulation. For use as the non-aqueous solution formulation and the suspension formulation, propyleneglycol, polyethylene glycol, vegetable oil such as olive oil, and an injectable ester such as ethylolate may be used. As a substrate for the suppository formulation, Witepsol, Macrogol, twin 61, cacao butter, laurin butter, or glycerogelatin may be used.

Cilostazol used in the present invention is conventionally used as a platelet coagulation inhibitor, a vasodilator, and a therapeutic agent for the treatment of ischemic peripheral blood vessel diseases. Accordingly, the safety of cilostazol is guaranteed. Although a dosage of the cilostazol may vary according to administration routes, severity of disease, gender, body weight and age, in general, the cilostazol dosage may be administered in an amount of 1.0 mg/kg to 5.0 mg/kg daily in a bolus or in multiple doses.

The pharmaceutical composition stated above may be administered to mammals such as rats, mice, livestock and humans via various routes. All of the administration methods are predictable, and for example, the dosage may be administered orally or rectally, or by intravenous, nasal, muscular, subcutaneous, intrauterine subdural, or intracerebroventricular injection.

### Advantageous Effects

According to the present invention, cilostazol inhibits expression levels of TNF-α and fatty acid synthase (FAS) genes in a concentration-dependent manner, and also significantly inhibits activity of caspase-3. Accordingly, cilostazol shows superior effects for the treatment or prevention of alcoholic liver diseases, in particular, alcoholic hepatitis compared to pentoxifylline which is conventionally used as a therapeutic agent for the treatment for alcoholic hepatitis. Thus, cilostazol is suitable for use as a drug for the treatment or prevention of alcoholic hepatitis.

### DESCRIPTION OF THE DRAWINGS

FIG. 1 shows the results of MTS assay to identify the protective effects of cilostazol on the viability of liver cells treated with ethanol,

FIGS. 2 and 3 show the inhibitory effects of cilostazol on caspase-3 activity in the liver cells treated with ethanol, evaluated by using a western blot and an activity assay kit,

FIG. 4 shows results of Hoechst staining assay to identify the inhibitory effects of cilostazol on liver cell apoptosis caused by ethanol,

FIG. 5 shows the inhibitory effects of cilostazol on LPS-stimulated TNF-α increase in RAW264.7 macrophage,

FIG. 6 shows results of LPS-induced ROS generation for different time in RAW264.7 macrophage,

FIG. 7 shows the effect of cilostazol on LPS-induced ROS production in RAW264.7 macrophage treated with ethanol,

FIG. 8 shows results of caspase-3 activity in liver when treated with ethanol in vivo for different time,

FIG. 9 shows the effects of cilostazol on the increased caspase-3 activity in liver when treated with ethanol in vivo,

FIG. 10 shows results of the expression of FAS gene in liver when treated with ethanol in vivo for different time, and

FIG. 11 shows effects of cilostazol on the increased FAS gene expression in liver when treated with ethanol in vivo.

### BEST MODE

The present invention provides a pharmaceutical composition including cilostazol represented in Formula 1 or a pharmaceutically acceptable salt thereof for the treatment and prevention of alcoholic liver diseases.

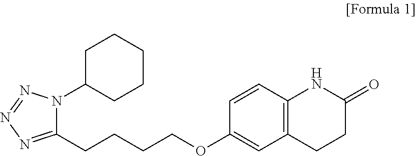 [SMILES: O=C1CCc2cc(OCCCCc3nnnn3C3CCCCC3)ccc2N1]

The alcoholic liver diseases may be alcoholic hepatitis.

The cilostazol or pharmaceutically acceptable salt thereof is a TNF-α inhibitor, which inhibits TNF-α induced by LPS, expression of fatty acid synthase FAS gene, and caspase-3 activity in vitro and in vivo.

The pharmaceutically acceptable salt of cilostazol according to the present invention may be in the form of an acid additive salt. For example, the pharmaceutically acceptable salt of cilostazol may be easily prepared by reacting cilostazol with a pharmaceutically acceptable acid.

The pharmaceutically acceptable acid may be, for example, an organic acid, such as an oxalic acid, a maleic acid, a fumaric acid, a malic acid, a tartaric acid, a citric acid, or a benzoic acid, or an inorganic acid, such as a hydrochloric acid, a sulfuric acid, a phosphoric acid, or a hydrobromic acid.

The cilostazol or pharmaceutically acceptable salt thereof may be included in an amount of 0.1 to 50 parts by weight based on 100 parts by weight of the pharmaceutical composition.

### MODE OF THE INVENTION

Hereinafter, the present invention is described in detail by referring to Examples. The examples are presented herein for illustrative purpose only.

### Example 1

### Effects of Cilostazol on Primary Cultured Liver Cells

1. Preparation of Primary Cultured Liver Cell

Liver cells were separated from Sprague-Dawley rats (8 to 10 weeks) or C57BL/6 (8 to 10 weeks) mouse via in situ collagenase perfusion, and then cultured in DMEM containing 10% FBS, 100 U/ml penicillin, 100 μg/ml streptomycin, 4 mM L-glutamine, and 100 nM dexamethasone. After 3 hours, the culture solution was replaced with DMEM including 0.1% FBS and 10 nM dexamethasone, and then, cultured for 16 hours (overnight).

For the treatment, the cells were treated with various concentrations of ethanol (0, 100, 200 mM) alone or together with cilostazol (Otsuka) or pentoxifylline which is used as a therapeutic agent for alcoholic hepatitis, and the resultant cell reactions were compared with each other. After the treatment with ethanol, a culture dish was sealed with a parafilm to prevent evaporation of ethanol.

2. Effects of Cilostazol on the Viability of Liver Cells Treated with Ethanol

MTS assay was performed in the following manner to identify effects of cilostazol on decreased liver cell viability caused by ethanol.

That is, the viability of liver cells was measured by using a MTS assay kit (Promega, Madison, Wis., USA). Liver cells were seeded on a collagen-coated 96 well plate (5×10⁻⁴ cell/well), and then pre-treated with cilostazol or pentoxifylline, followed by the treatment with ethanol for 21 hours. An MTS solution were added to the respective wells and then, the cells were incubated at 37° C. for 4 hours, and absorption of the result was measured at a wavelength of 490 nm.

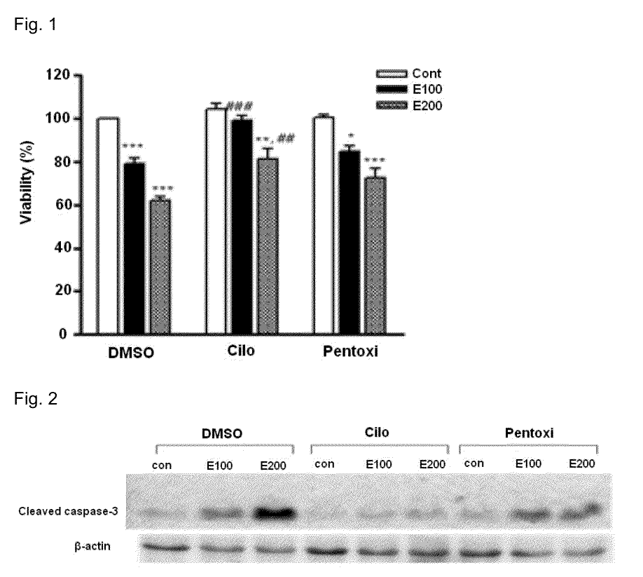

Referring to FIG. 1, in DMSO-treated group, the liver cell viability was decreased by ethanol (100, 200 mM) treatment for 24 hour in concentration-dependent manner, which was significantly recovered by pretreatment with cilostazol (100 μM). On the other hand, pretreatment with pentoxifylline (100 μM) did not significantly improve the cell viability compared with the DMSO group.

3. Effects of Cilostazol on Caspase-3 Activity Increased by Ethanol

Caspase-3 activity was measured by Western blotting with cleaved caspase-3 antibody.

That is, liver cells were treated with ethanol for 24 hours, and then, lysed in a lysis buffer (10 mM HEPES, pH 7.4, 10 mM b-glycerophosphate, 1 mM EDTA, 1 mM EGTA, 1 mM Na₃VO₄, 2 mM MgCl₂, 1 mM DTT, 1 mM PMSF, 1 mM benzamidin, 10 μg/ml aprotinin, 10 μg/ml leupeptin, 10 μg/ml pepstatin A, 1% NP-40), and then the protein was subjected to electrophoresis in 15% SDS-PAGE gel and transferred onto a nitrocellulose membrane (NC). NC was incubated with a cleaved caspase-3 antibody (cell signaling, Beverly, Mass.) at 4° C. for 16 hours, and then, with horseradish peroxidase conjugated rabbit IgG antibody at room temperature for 1 hour. The protein bands were developed with chemiluminescence detection system, and then a protein band was detected with LAS-3000 (FUJI FILM) (European Journal of Pharmacology 508 (2005) 31-45).

Referring to FIG. 2, in DMSO treated control, ethanol increased the activity of caspase-3 in concentration-dependent manner, and the caspase-3 activity was reduced by the pretreatment with cilostazol (100 μM) and pentoxifylline (100 μM), where cilostazol showed better inhibitory effects than pentoxifylline.

Also, the caspase-3 activity was measured by using an activity assay kit (R&D kit). That is, caspase-3 activity was measured by using a caspase-3 colorimetric assay kit (R&D system, Minneapolis, Minn.). Liver cells were lysed by using lysis buffer, and then the protein (150 μg) was incubated with DEVD-pNA, a caspase-3 substrate at 37° C. for 2 hours, and then absorption of the product was measured at a wavelength of 405 nm. A standard curve was obtained by using recombinant human caspase-3 protein and a caspase-3 activity was calculated as ng/mg protein, and results thereof were shown as fold increase of activity (Cancer letters 270 (2008) 40-55).

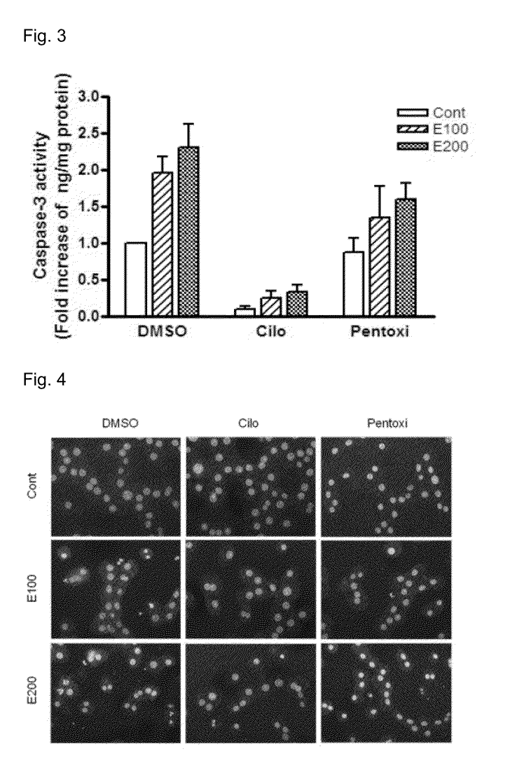

Referring to FIG. 3, in DMSO treated control, ethanol concentration-dependently increased caspase-3 activity in liver cells, and when the liver cells were pretreated with cilostazol, the caspase-3 activity was almost completely inhibited. Pentoxifylline also reduced caspase-3 activity caused by ethanol, but the inhibitory effect of pentoxifylline was smaller than that of cilostazol. This result was similar to that of western blotting.

4. Effects of Cilostazol on Liver Cell Apoptosis Caused by Ethanol

Nuclei of liver cells were stained with Hoechst to measure cell apoptosis. That is, liver cells cultured on collagen coated glass cover slips were fixed with an ice-cold methanol/acetic acid (3:1), and then stained with Hoechst 33342 (5 μg/ml). After washing with distilled water, the cover slip was mounted in glycerol containing 50% glycerol including 20 mM citric acid and 50 mM Na₂HPO₄ and apoptotic nuclei were identified under a fluorescent microscope (European Journal of Pharmacology 508 (2005) 31-45).

Referring to FIG. 4, nuclear fragmentation caused by ethanol was reduced by pretreatment with cilostazol.

### Example 2

### Effects of Cilostazol on RAW264.7 Macrophage

1. Preparation of RAW264.7 Macrophage

RAW264.7 cells were obtained from Korean Cell Line Bank and cultured in a DMEM solution containing 10% FBS, 100 U/ml penicillin, and 100 μg/ml streptomycin for use in experiments. To compare direct effects of ethanol with indirect effects caused by endotoxin, effects of LPS were identified.

2. Effects of Cilostazol on LPS-Stimulated TNF-α Production in RAW264.7 Macrophage

RAW264.7 macrophage prepared as described above was treated with LPS (50 ng/ml) for 4 hours, and then, TNF-α level in the cell culture was measured by using ELISA kit (R&D).

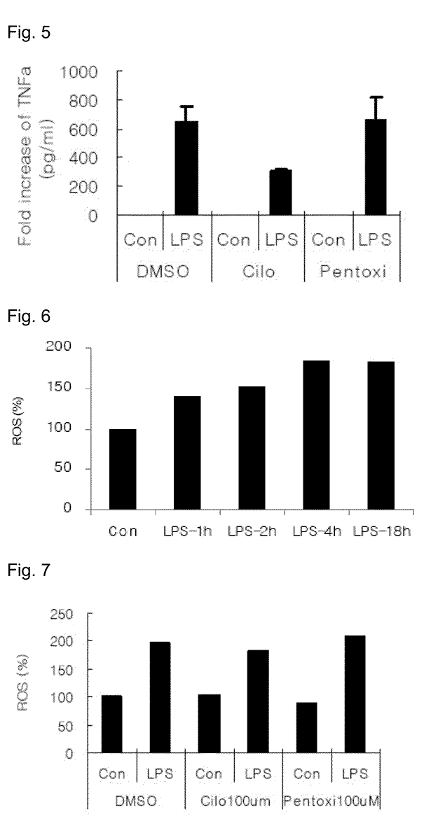

Referring to FIG. 5, in the DMSO treated control group, the TNF-α level in culture media was increased about 700 times by LPS, which was decreased by ˜50% by pretreatment with cilostazol (p=0.016). On the other hand, pentoxifylline did not show significant inhibitory effects. Accordingly, cilostazol more efficiently inhibited the release of TNF-α than pentoxifylline, a therapeutic agent for alcoholic hepatitis.

3. Effects of Cilostazol on LPS-Induced ROS Increase in RAW264.7 Macrophage

RAW264.7 macrophage prepared as described above was treated with LPS (50 ng/ml) for 0 to 18 hours. Separately, RAW264.7 macrophage prepared as described above was pretreated with cilostazol (100 μM) or pentoxifylline (100 μM) for 1 hour and then, treated with LPS (50 ng/ml) for 4 hours. The RAW264.7 macrophages were treated with H₂DCFDA (50 μM) for 40 minutes, and then, the production of ROS was evaluated by using FACS.

Referring to FIG. 6, ROS production in cells was increased by LPS (50 ng/ml) with time, and the maximum increase (about 2 times) reached at 4 hours. However, as shown in FIG. 7, the pretreatment with neither cilostazol nor pentoxifylline significantly inhibited the production of ROS induced by LPS. Accordingly, it was considered that TNF-α inhibitory effect of cilostazol is not mediated by the suppression of ROS production.

### Example 3

### Effects of Cilostazol on Alcohol-Induced Liver Injury in Animal Model

1. Preparation of Alcohol-Induced Acute Liver Injury Animal Model

Eight-week old mice were subjected to binge alcohol drinking to induce liver damage. That is, mice were administered orally 6 g/kg of ethanol once. Cilostazol was orally administered in doses of 50 mg/kg/day and 100 mg/kg/day for 4 days before ethanol administration, and ethanol was orally administered 1 h after the last cilostazol administration. Then, animals were sacrificed at different time after ethanol administration.

2. Effects of Cilostazol on Caspase-3 Activity

Effects of cilostazol on caspase-3 activity were identified by using a caspase-3 activity assay kit (R&D) in the same manner as described above.

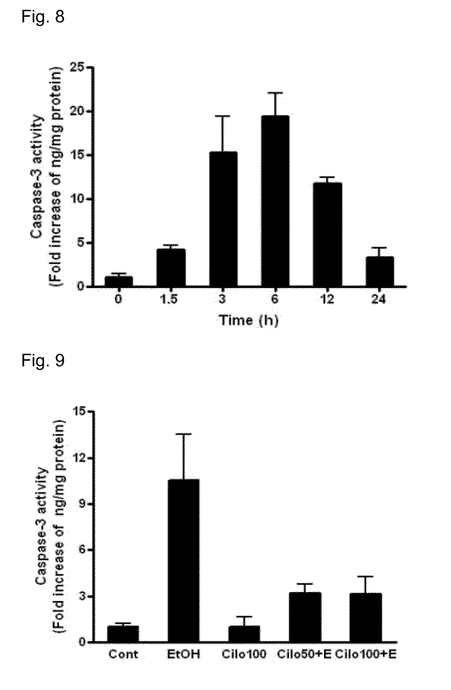

As shown in FIG. 8, caspase-3 activity increased to the greatest level (up to 20 times) at 6 hour after ethanol (6 g/kg) administration, and as shown in FIG. 9, caspase-3 activity was significantly reduced by oral administration of cilostazol (100 mg/kg).

3. Effects of Cilostazol on the Expression of FAS Gene

Effects of cilostazol on the expression of FAS gene were identified by real-time PCR assay. That is, total RNA was extracted from liver tissues by using a trizol reagent, and then 1 μg of total RNA was reverse-transcribed into cDNA by using high-performance cDNA reverse transcription kit (Applied Biosystems, Foster City, Calif., USA). The PCR reaction was performed such that cDNA, the respective primers were reacted with power SYBR Green PCR master mix (Applied Biosystems) by using real-time PCR 7500 software system (Applied Biosystem). The PCR conditions were initial incubation at 95° C. for 10 minutes, followed by 45 cycles of 95° C. for 15 seconds, 55° C. for 20 seconds, and 72° C. for 35 seconds.

A primer sequences used herein were constructed based on NCBI nucleotide DB by using Primer Express program (Applied Biosystems). β-actin [SEQ ID NO: 1(98 bp: forward, 5′-TAC TGC CCT GGC TCC TAG CA-3′); SEQ ID NO: 2 (reverse, 5′-TGG ACA GTG AGG CCA GGA TAG-3′)], FAS [SEQ ID NO: 3(76 bp: forward, 5′-CTG CGG AAA CTT CAG GAA AT-3′); SEQ ID NO: 4(reverse, 5′-TGT CAC TCC TGG ACT TGG G-3′)]. FAS mRNA level was normalized to β-actin mRNA level, and shown in fold increase.

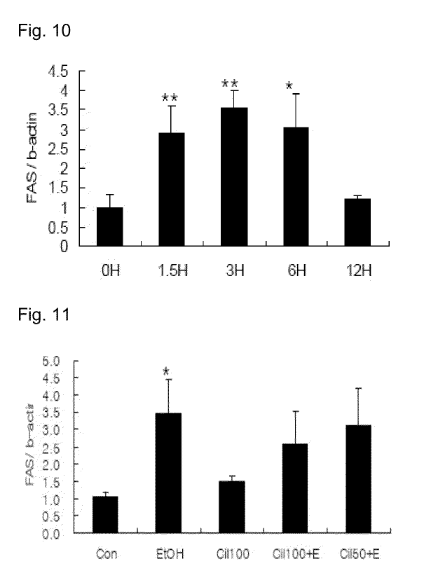

As shown in FIG. 10, the expression of FAS gene was maximized (up to 3.5 times) at 3 hours after the administration of ethanol, and as shown in FIG. 11, the expression of FAS gene was reduced by about 30% by oral administration of cilostazol (100 mg/kg).

Hereinafter, Preparation Examples of the pharmaceutical composition including cilostazol according to the present invention are presented. However, they are provided herein for illustrative purpose only.

### Preparation Example 1

### Preparation of Powder Formulation

20 mg of cilostazol, 100 mg of lactose, and 10 mg of talc were mixed and a sealing pack was filled with the mixture to prepare a powder formulation.

### Preparation Example 2

### Preparation of Tablet Formulation

20 mg of cilostazol, 100 mg of corn starch, 100 mg of lactose, and 2 mg of magnesium stearate were mixed and the mixture was subjected to tableting according to a conventional tablet preparation method, thereby forming a tablet formulation.

### Preparation Example 3

### Preparation of Capsule Formulation

10 mg of cilostazol, 100 mg of corn starch, 100 mg of lactose, and 2 mg of magnesium stearate were mixed according to a conventional capsule preparation method, and then, a gelatin capsule was filled with the mixture to prepare a capsule formulation.

### Preparation Example 4

### Preparation of Injection Formulation

10 mg of cilostazol, an appropriate amount of injectable sterilized distilled water, and an appropriate amount of a pH controller were mixed, and then, a conventional injection formulation preparation method was performed to make such amounts of components be included in each ample (2 ml).

### Preparation Example 5

### Preparation of Spray Formulation

HFA-227 was added to 0.08 wt % of cilostazol and 0.005 wt % of oleic acid in such an amount that the total amount of the components was 100 wt %, thereby preparing an aerosol suspension.

### INDUSTRIAL APPLICABILITY

According to the present invention, cilostazol inhibits expression levels of TNF-α and FAS (fatty acid synthase) gene in a concentration-dependent manner, and also significantly inhibits the activity of caspase-3. Accordingly, cilostazol shows superior effects for the treatment or prevention of alcoholic liver diseases, in particular, alcoholic hepatitis compared to pentoxifylline which is conventionally used as a therapeutic agent for the treatment for alcoholic hepatitis. Thus, cilostazol is suitable for use as a drug for the treatment or prevention of alcoholic hepatitis. Accordingly, cilostazol can be used in various industrial fields including hospitals and research institutes.

### [Sequence List Pretext]

Sequence 1 indicates a forward primer for beta-actin.

Sequence 2 indicates a reverse primer for beta-actin.

Sequence 3 indicates a forward primer for FAS.

Sequence 4 indicates a reverse primer for FAS.

1. A pharmaceutical composition for the treatment and prevention of alcoholic liver disease, comprising cilostazol represented in Formula 1 below or a pharmaceutically acceptable salt thereof:

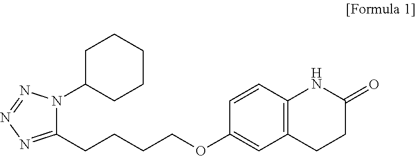 [SMILES: O=C1CCc2cc(OCCCCc3nnnn3C3CCCCC3)ccc2N1]

2. The pharmaceutical composition of claim 1, wherein the alcoholic liver disease is alcoholic hepatitis.

3. The pharmaceutical composition of claim 1, wherein the cilostazol or pharmaceutically acceptable salt thereof is a TNF-α inhibitor.

4. The pharmaceutical composition of claim 1, wherein the pharmaceutically acceptable acid is in the form of an acid addition salt prepared by using an organic acid selected from the group consisting of an oxalic acid, a maleic acid, a fumaric acid, a malic acid, a tartaric acid, a citric acid, and a benzoic acid; or an inorganic acid selected from the group consisting of a hydrochloric acid, a sulfuric acid, a phosphoric acid, and a hydrobromic acid.

5. The pharmaceutical composition of claim 1, wherein the cilostazol or pharmaceutically acceptable salt thereof is included in an amount of 0.1 to 50 parts by weight based on 100 parts by weight of the pharmaceutical composition.


SEQUENCE LISTING (4 sequences)

SEQ ID NO: 1 - DNA, 20 bp, Artificial Sequence
  Forward primer for beta-actin
tactgccctggctcctagca

SEQ ID NO: 2 - DNA, 21 bp, Artificial Sequence
  Reverse primer for beta-actin
tggacagtgaggccaggatag

SEQ ID NO: 3 - DNA, 20 bp, Artificial Sequence
  Forward primer for FAS
ctgcggaaacttcaggaaat

SEQ ID NO: 4 - DNA, 19 bp, Artificial Sequence
  Reverse primer for FAS
tgtcactcctggacttggg